(19) United States
(12) Patent Application Publication
(10) Pub. No.: US 2026/0007868 A1
(43) Pub. Date: Jan. 8, 2026

(54) DILATOR TIP DESIGN FOR ENHANCED FORCE TRANSMISSION

(71) Applicant: BIOTRONIK AG, Buelach (CH)

(72) Inventors: Marc Gianotti, Wiesendangen (CH); Dragana Margeta, Stetten (CH); Ulf Fritz, Bargen (CH); Michael Jetter, Thayngen (CH); Valentin Nickel, Singen-Schlatt (DE); Lucien Pistol, Rielasingen-Worblingen (DE)

(21) Appl. No.: 18/997,232

(22) PCT Filed: Aug. 8, 2023
(86) PCT No.: PCT/EP2023/071928

§ 371 Date: Jan. 21, 2025

(30) Foreign Application Priority Data
Aug. 9, 2022 (EP) 22189373.8

(51) Int. Cl. A61M 29/02; A61M 25/00

(52) U.S. Cl. CPC A61M 29/02; A61M 25/005; A61M 25/0082

(57) ABSTRACT

A dilator including a shaft and a reinforcing element. The reinforcing element is connected to a distal end of the shaft. The reinforcing element is in contact with the distal end of the dilator shaft and the connection element. The connection element at least in part encloses the dilator shaft and/or the reinforcing element. The connection element extends into at least one indentation recess or through hole of the reinforcing element. The connection element consists of a polymer. The reinforcing element consists of a metal or metal alloy.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/071928, which was filed Aug. 8, 2023, which application claimed priority from EP Application 22189373.8, which was filed Aug. 9, 2022.

### FIELD OF THE INVENTION

A field of the invention concerns dilators used in catheter systems, which can be used to treat stenosis or lesions caused by atherosclerotic diseases.

### BACKGROUND

Catheters have been used in many ways to inspect or treat vessels. Atherosclerosis is a vascular disease, which is manifested by the accumulation of degenerative material along the of blood vessel walls. The affected blood vessels can develop plaques/lesions, which can cause a successive reduction of a vessel lumen diameter and thus a restriction in blood flow. A chronic plaque build-up (i.e., composed of mixtures of fatty, fibrous and/or calcified tissue matters) can lead to a chronic total occlusion (CTO).

### SUMMARY OF THE INVENTION

A preferred dilator has a dilator shaft, a reinforcing element which is connected to a distal end of the dilator shaft and a connection element. The reinforcing element is in contact with the distal end of the dilator shaft and the connection element. The connection element at least in part encloses the dilator shaft and/or the reinforcing element. The connection element extends into at least one indentation, recess or through hole of the reinforcing element. The connection element consists of a polymer. The reinforcing element consists of a metal or metal alloy. Preferably, the dilator shaft is a braided dilator shaft (i.e. a dilator shaft including a reinforcing braid, e.g. made of a polymer, metal and/or metal alloy).

### BRIEF DESCRIPTION OF THE DRAWINGS

In the following, detailed exemplary embodiments will be described with reference to the following drawings showing in

FIG. 1 a longitudinal cross-section of an exemplary dilator;

FIG. 2 a longitudinal cross-section of a further exemplary dilator;

FIGS. 3A, 3B, 3C an exemplary reinforcing element;

FIGS. 4A, 4B further exemplary dilators;

FIG. 5 an aspect concerning the manufacturing of an exemplary dilator;

FIG. 6 a further aspect concerning the manufacturing of an exemplary dilator;

FIGS. 7A, 7B a further aspect concerning the manufacturing of an exemplary dilator;

FIG. 8 a further aspect concerning the manufacturing of an exemplary dilator;

FIG. 9 a further aspect concerning the manufacturing of an exemplary dilator;

FIG. 10 a further aspect concerning the manufacturing of an exemplary dilator,

FIG. 11 shows an embodiment of a catheter system,

FIG. 12 shows an embodiment of a support catheter,

FIG. 13 shows an embodiment of a dilator.

### DETAILED DESCRIPTION OF THE PREFERRED EMBODIMENTS

When the dilator shaft is moved towards a stenosis (e.g., a CTO), the reinforcing element may contact with the stenosis. Due to the increased force resistance that the reinforced element provides compared to the dilator shaft as such (e.g. reduced tendency to bend, break and/or kink), a higher force may be transferred onto the stenosis by movement of the dilator shaft. Preferred dilators provide a structure that separates functions between the dilator shaft and the reinforcing element. The reinforcing element selectively reinforces the distal end of the dilator shaft. Hence, force transmission to, for example, and the risk of kinking, e.g. if there is a non-symmetric force applied to the reinforcing element, e.g. by a non-symmetric stenosis surface, is reduced. In particular, a direct force transmission from the dilator shaft to the reinforcing element and to the stenosis can be facilitated.

The reinforcing element may include a proximal portion and a distal portion being situated distal to the proximal portion. The proximal portion may have (generally) cylindrical shape. The (generally) cylindrical proximal portion forms an outer proximal side and an inner proximal side. The proximal portion includes at least one indentation, recess or through hole, preferably several (i.e. two, three or more than three) through holes. The distal portion may be distally tapered. Preferably, the reinforcing element includes a cylindrical proximal portion and a conical distal portion being situated distally to the cylindrical proximal portion. The distal portion has an outer diameter (e.g. 0.9 to 1.3 mm). The outer diameter of distal portion at its proximal end may be larger than an outer diameter of (cylindrical) proximal portion. The outer diameter of distal portion may taper distally. For example, at a distal end of distal portion its outer diameter may be similar to or larger than an outer diameter of (generally) cylindrical outer side. In one embodiment the reinforcing element may have a distal opening and/or a lumen running through its proximal and/or distal region. In such a case a guidewire or another medical device can run through the lumen. In another embodiment the proximal and/or the distal portion of the reinforcing element include no lumen(s) and/or is solid. In such a case, the reinforcing element can be used for dottering into a stenosis. The distal end of the distal portion of the reinforcing element may be rounded and/or blunt. The distal end of the distal portion of the reinforcing element may form the very distal end of the dilator.

For example, the dilator shaft and the reinforcing elements may include different materials and/or may be fabricated separately and subsequently connected. This also allows full design flexibility in terms of material selection and fabrication methods for the separate components. For example, the reinforcing element may include a material that has a higher hardness than a material (or any material) included by the dilator shaft. By means of a (generally) cylindrical and/or conical metal or metal alloy reinforcing element, a particularly strong connection may be feasible.

The dilator may be used within a catheter system. For examples, the dilator may be used together with a multifunctional catheter system, preferably a peripheral multifunctional catheter system.

The multi-functional catheter system includes a retractable sheath which can accommodate consecutively different inner members, like a dilator, a guidewire and/or balloon catheter (e.g. PTA or PTCA catheter). Thus a (peripheral) multi-functional catheter system including a retractable sheath, preferably a support catheter, which enables consecutive accommodation of more than one inner member, wherein the multi-functional catheter systems includes as one of the inner members a dilator according as described herein is disclosed as well.

The peripheral multifunctional catheter system may be an intravascular balloon catheter system, supplied with a retractable sheath (e.g. support catheter), a flexible catheter (e.g. dilator) and a Percutaneous Transluminal Angioplasty balloon (PTA balloon). The support catheter and the dilator are used in tandem to facilitate lesion access and lesion crossing. The peripheral multifunctional catheter system allows a variable guide wire support and injection of fluids, and adjustable length inflatable balloon (e.g. up to 180 mm).

Such a multi-functional catheter system enables guidance and support of a guidewire during access and crossing of lesions in the vasculature. It further allows for wire exchanges and provide a conduit for the delivery of saline solutions or diagnostic contrast agents to facilitate wire crossing.

Thus, a catheter system, preferably a multi-functional catheter system, including a dilator as described above is disclosed herein as well. The catheter system may further include a support catheter. The support catheter has a support catheter shaft defining a support catheter lumen capable of receiving the dilator and optionally a balloon catheter. The support catheter may have at its distal portion a metal ring. The metal ring may be spaced apart from the distal end of the support catheter. The dilator shaft may be reinforced e.g. with a metal braid. The support catheter shaft may be reinforced with a metal braid or a metal braid with interlaced pairs of longitudinally wires.

The use of such a catheter system for crossing a stenosis or a chronic total occlusion is described herein as well.

Also the use of such a catheter system as a crossing catheter is described herein.

Further described herein is a method of treating a stenosis or a chronic total occlusion with a dilator and/or a catheter system as described above.

It is noted that the first aspect may not only be helpful for treating stenosis but also for a variety of further intravascular applications. The design according to the first aspect may be combined with any dilator technology, for example where the tip needs to be durable when longitudinal forces are transferred. The distal dilator tip preferably includes a polymer part and a metal part.

For example, the reinforcing element may at least in part extend distally with respect to a distal end of the dilator shaft. The reinforcing element may be adapted to form a distal-most part of the dilator. Hence, the reinforcement may particularly be present distally at the dilator, where often the strongest forces arise.

The dilator may further include a connection element which at least in part encloses the dilator shaft and/or the reinforcing element. The connection element may be adapted to connect the dilator shaft and reinforcing element to each other. For example, the connection element may form an atraumatic tip of the dilator. For example, the connection element may include a material that is softer and/or more flexible compared to a material of the dilator shaft and/or the reinforcing element. Hence, when contacting a vessel wall, for example, the risk of penetration or dissection may be reduced.

In some examples, the shaft may be adapted to be relatively flexible in its distal-most portion (compared to a more proximal portion of the dilator shaft), e.g. to allow steering the distal tip away from a vessel wall.

The reinforcing element may at least in part extend distally with respect to a distal end of the connection element. Hence, the reinforcing element may form a reinforced tip of the distal dilator end, e.g., supporting treatment of stenosis, while the tip may be followed proximally by a relatively softer material of the connection element. Additionally or alternatively, the connection element may have an extension such as to reach a larger outer diameter compared to an outer diameter (or any diameter) of the reinforcing element.

Hence, the delicate, reinforced tip may by surrounded distally and/or radially by relatively softer material reducing risks of damaging vessel walls. The dilator may have an atraumatic tip (e.g., provided by the connection element) with a locally reinforced strength (e.g., provided by the reinforcing element).

An exemplary longitudinal extension from a distal-most part of the reinforcing element to a proximal-most part of the connection element is within the range 1.0 to 20.0 mm, 2.0 to 10.0 mm, e.g. 6.0 to 7.0 mm. A relatively long extension may be beneficial in some aspects, e.g. for crossing elongate lesions. However, a downside is that a long extension would reduce maneuverability and be possibly more traumatic and/or lead to increased friction. The specified values may represent an optimum compromise in this regard.

The portion of the reinforcing element extending distally with respect to the distal end of the connection element may also be referred to as distal portion of the reinforcing element.

In some examples, the reinforcing element may have a longitudinal extension of 0.1 to 5.0 mm, or 0.5 to 3.0 mm. For example, the distal portion of the reinforcing element may have a length (longitudinal extension) of 1.0 mm to 2.0 mm, e.g. 1.5 mm, and the proximal portion of the reinforcing element may have length (longitudinal extension) of 1.0 mm to 2.0 mm, e.g. 1.5 mm.

For example, the distal portion of the reinforcing element may support anchoring of the atraumatic tip (e.g. in a stenosis, such as a CTO).

The connection element may include a polymer. Polymer materials may be particularly suitable to provide a relatively soft connection element which is at the same time durable, biocompatible and wear-resistant. Further, polymer materials may allow efficient manufacturing methods, such as injection molding. In some examples, the connection element is injection molded.

The reinforcing element may include at least one indentation, recess or through hole. For example, the (generally) cylindrical proximal portion of the reinforcing element, e.g. an inner and/or an outer side of the reinforcing element, may include at least one indentation. In some examples, the at least one indentation may include at least one through hole. In other examples, the at least one indentation may include at least one partial indentation, e.g. a hole that only partly extends into an outer side of the reinforcing element, e.g. without penetrating the reinforcing element.

The connection element may extend at least in part into at least one indentation, recess or through hole of the reinforcing element. For example, the at least one indentation, e.g. at the (generally) cylindrical proximal portion of the reinforcing element, may be a least partly filled by the connection element. This may ensure a particularly tight connection between the connection element and the reinforcing element. Particularly, this may provide a tight connection despite longitudinal forces acting on the connection element and/or the reinforcing element.

It is noted that additionally or alternatively, the dilator shaft may include at least one indentation, e.g. in a (generally) cylindrical outer side of the dilator shaft. The at least one indentation may be designed as described with reference to the at least one indentation of the reinforcing element. The connection element may extend at least in part into the at least one indentation of the dilator shaft, e.g. such as to render a connection between the connection element and the dilator shaft particularly strong.

The connection element may be tapered in a distal and/or proximal direction. This may ensure a particularly tight connection between the connection element and the dilator shaft and/or the reinforcing element, e.g., as the thickness and thus stability of the connection element may be increased at a proximal end of the reinforcing element. At the same time, particularly a distal tapering may improve the maneuverability of the dilator within a vessel.

For example, a distal and/or proximal portion of the connection element may, when seen in a longitudinal cross-section form a wedge with an angle between 3° and 30°, or 5 and 25°.

The connection element may extend at least in part around an outer diameter of the dilator shaft and, for example, an outer diameter of the reinforcing element, e.g. a (generally) cylindrical outer side of the reinforcing element, in particular such as to form a step. For example, at the step, an inner diameter of the connection element may change such that a transition zone is provided between a portion with a relatively larger inner diameter and a portion with a relatively smaller inner diameter. Hence, the dilator shaft and/or the reinforcing element, e.g. its (generally) cylindrical proximal portion or outer side may be provided with an additional anchoring at the transition zone. For example, when the portion with a relatively larger inner diameter is arranged distally and the portion with a relatively smaller inner diameter is arranged proximally, the transition zone may provide additional support resisting a relative longitudinal movement of the dilator shaft and/or the reinforcing element relative to the connection element.

The (generally) cylindrical proximal portion of the reinforcing element, e.g. a (generally) cylindrical inner side of the reinforcing element, may for example at least in part, extend around an outer diameter of the dilator shaft. For example, the (generally) cylindrical proximal portion of the reinforcing element at least in part be glued, injection molded or otherwise connected to an outer surface of the shaft. In some examples, the connection element may connect the (generally) cylindrical proximal portion of the reinforcing element to the dilator shaft.

If the (generally) cylindrical proximal portion of the reinforcing element extends at least in part around an outer diameter of the dilator shaft, an inner lumen of the dilator shaft may be at least in part unaffected by the additional reinforcing element. For example, a guidewire may be moved within the lumen without being obstructed by the reinforcing element. Moreover, a low profile dilator may be provided that may be easily manufactured, e.g. by injection molding of the connection element around the dilator shaft and the reinforcing element.

In some examples, the (generally) cylindrical proximal portion of the reinforcing element at least in part extends along an inner diameter of the dilator shaft. For example, the (generally) cylindrical proximal portion of the reinforcing element, e.g. forming a (generally) cylindrical outer side, may be glued or otherwise connected to an inner surface of the dilator shaft. Also in these example, injection molding of the connection element around the dilator shaft and the reinforcing element may be used.

The dilator shaft may include a distal region with the same inner diameter and a proximal region with a same inner diameter than the inner diameter of the reinforcing element. This may allow increased functionality at the distal region allowing more space to accommodate potential functions.

The reinforcing element (e.g. it is generally cylindrical outer side) may end or abut at a transition zone (e.g. a shoulder) between the distal region and the proximal region of the dilator shaft. Hence, the reinforcing element may be additionally anchored and relative movement of the reinforcing element and the dilator shaft in longitudinal direction may be particularly inhibited. For example, the dilator shaft may include, e.g. in the transition zone, an at least partly radially extending portion which may absorb longitudinal forces (similarly as described herein with reference to a step of the connection element). The direct force transmission from the dilator shaft to the reinforcing element and then to the stenosis may hence be further improved.

The connection element may at least in part extend around the distal region and the proximal region of the dilator shaft such as to form a step. The step may thus further reinforce the anchoring of the dilator shaft, the reinforcing element and the connection element, particularly in a longitudinal direction. The connection element may have an olive shape. Olive shape means the mid portion of the connecting element has a larger outer diameter and/or circumference than the distal portion and the proximal portion of the connecting element. The olive shape can be seen mathematically as an ellipsoid (wherein the longer side is oriented along the longitudinal dilator axis). The olive shape enables an evenly dispersion of injected contrast agent over the lesion. The olive shape also enables low volume contrast injections (puffs) for optimal visualization due to the improved liquid flow around the dilator tip.

The connection element may have at its mid portion an outer diameter of 0.5 mm to 3.0 mm, e.g. 1.0 mm to 2.0 mm.

The reinforcing element may include or consist of a metal or metal alloy. Hence, a particularly strong reinforcement may be provided. The metal or metal alloy reinforcing element increases the dilator's durability, visibility and to withstand loads during clinical use without deformation.

For example, stainless steel, titanium and/or other biocompatible metals or metal alloys may be used. Also, for example suitable alloys, e.g. Nitinol, etc. may be used. The dilator shaft, for example, may include a polyamide (e.g. PA 12) or a polyether-block-amide.

In other examples, the reinforcing element may include a polymer and/or ceramic material. For example, the reinforcing element may include a polymer which has a higher hardness, stiffness, and/or bending resistance, etc. compared to a material of the connection element (e.g. a polymer of the connection element) and/or the dilator shaft (e.g. a polymer of the dilator shaft).

The reinforcing element may include a distal opening. Hence, there may be functional communication between an outside of the dilator and an inside of the dilator via the reinforcing element. For example, a guidewire may be extended from an inner lumen of the dilator to a region distally from the dilator. This may allow particular compatibility with dilators that require the mentioned functional communication.

For example, the dilator (e.g. the dilator shaft) may include an inner lumen for accommodating a guidewire, such that the guidewire may be moved distally through the lumen such that the guidewire extends out of the distal end of the dilator.

For example, a diameter of the distal opening may be smaller than an inner diameter of the (generally) cylindrical outer surface. Hence, the reinforcing element may include a portion that at least in part extends radially (e.g. from the relatively narrow opening to the relatively wider (generally) cylindrical outer surface) which may improve force transmission in longitudinal direction.

In other examples, for example, the distal opening may include a diameter that is essentially given by an inner diameter of the (generally) cylindrical outer surface. For example, the reinforcing element may include a shape similar to an open cylinder at its distal side. This may be helpful for anchoring, as, e.g., the relatively narrow cylindrical walls may cut into a possible stenosis, helping to remove it.

In some examples, the reinforcing element includes a tapered distal end. This may increase maneuverability when moving through a vessel and/or a stenosis. The reinforcing element may also improve anchoring. An opening may be provided at the distal end, for example.

In some examples, the distal opening may be provided in a tapered region of the reinforcing element that may be provided at a distal side of the reinforcing element. The tapered region may be followed, proximally, e.g. by a (generally) cylindrical element. For example, the reinforcing element may thus have a (generally) cylindrical inner side with e.g. a constant inner diameter. The (generally) cylindrical element may also include a (generally) cylindrical outer side having, for example, a constant outer diameter.

It is noted that the dilator may also be adapted such that the reinforcing element does not include a distal opening, e.g. the reinforcing element may include a closed distal surface. Particularly in this case, the dilator may be provided without lumen and/or guidewire functionality.

Concerning the term generally cylindrical proximal portion (e.g. surface) of the reinforcing element as outlined herein, it is noted that the reinforcing element does not only include cylindrical surfaces, but also for example: cylindrical surfaces with one or more openings, surfaces that follow a generally cylindrical shape but include local variations (for example, a generally circular cross section may be modulated by a small, e.g. sinusoidal, fluctuation, or a generally circular cross section may be approximated by a polygon, etc.). It is noted that the elements described herein as having a generally cylindrical proximal portion may include a generally cylindrical inner face and/or a generally cylindrical outer face.

A method for manufacturing a dilator may include providing a dilator shaft and a reinforcing element having a (generally) cylindrical proximal portion. The method may further include connecting the reinforcing element to a distal end of the dilator shaft.

In some example, the method may include a step of injection molding of a connection element such that the connection element at least in part encloses the dilator shaft and/or the reinforcing element. This may allow a particularly efficient method for manufacturing the dilators described herein.

It is noted that whenever reference is made to A “includes” B herein, it is understood that also the option A (essentially) consisting of B is included.

Moreover, it is noted that all steps described herein with respect to a method are understood to also disclose the corresponding dilator and the properties implied by the method. Similarly, all aspects described herein with respect to a dilator are understood to also include the corresponding method steps.

Although not expressly stated in all instances, the elements described hereinbelow may generally include the features as described above with respect to similarly designated elements and vice versa. Also, not each and every aspect is repeated with reference to each Figure. Hence, the embodiments outlined below may include features described with reference to other embodiments or more generally hereinabove, unless the contrary is explicitly indicated.

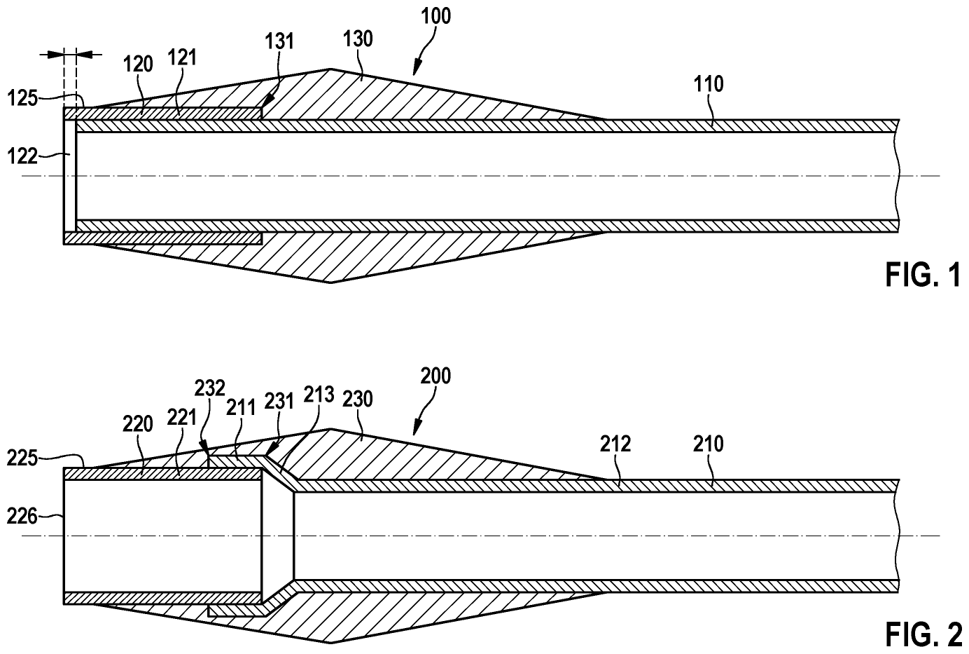

FIG. 1 shows a longitudinal cross-section of an exemplary dilator 100 which may include a dilator shaft 110, a reinforcing element 120 and a connection element 130.

Dilator shaft 110 has a (generally) cylindrical shape, particularly an elongated cylindrical shape, such as tubular shape. In some examples, dilator shaft 110 may include a reinforced material. For example, dilator shaft 110 may include a braided material, such as a braided metal. In some examples, dilator shaft 110 may be fiber-reinforced.

Reinforcing element 120 may include a (generally) cylindrical element 121 that forms (generally) cylindrical faces in the form of inner and outer sides of reinforcing element 120. The cylindrical element 121 may include a constant inner and outer diameter. In other examples, a variable inner and/or outer diameter may be used. The reinforcing element 120 may further include a closed distal side 122. A thickness of the closed distal side 122 may be the same or essentially similar to a thickness of (generally) cylindrical element 121. The reinforcing element 120 may include a (generally) cylindrical cap-like shape. As shown in FIG. 1, distal end of dilator shaft 110 may be in contact with an inner side of closed distal side 122.

(generally) cylindrical element 121 may include an inner diameter such as to fit onto an outer diameter of dilator shaft 110 at a distal portion of dilator shaft 110. (generally) cylindrical element 121 may for example be clipped on the outer diameter of dilator shaft 110, the cylindrical element may be shrink-fitted, pressed onto the outer diameter of dilator shaft 110, and/or connected via an adhesive etc.

In some examples, distal side 122 may include a (central) opening (not shown in FIG. 1), such as to provide fluid communication between an inner lumen of dilator shaft 110 and an exterior of dilator 100. For example, a guidewire may then be used which is guided inside the (reinforced) dilator shaft 110.

Connection element 130 may extend around a proximal portion of (generally) cylindrical element 121 (or: its (generally) cylindrical outer side). A distal portion 125 of (generally) cylindrical element 121 may be free from connection element 130. Connection element 130 may include a variable outer diameter. For example, the outer diameter may get smaller towards a distal end (e.g. in a distally tapered portion) and a proximal end (e.g. in a proximally tapered portion) of connection element 130, e.g. in a symmetric manner. The outer diameter may include a maximum at a center portion of connection element 130. The connection element may include or essentially consist of a polymeric material, e.g. injection molded over reinforcing element 120 and dilator shaft 110.

Connection element 130 may extend along an outer diameter of dilator shaft 110 and of reinforcing element 120. At a proximal end of reinforcing element 120, connection element 130 may form a step 131.

A longitudinal extension of reinforcing element 120 (or (generally) cylindrical element 121 or its inner and/or outer sides) may include a value that is 1-10 times an inner radius of dilator shaft 110, for example 2-8 times, or 3-6 times.

A longitudinal extension of connection element 130 may include a value that is 1-10 times the longitudinal extension of (generally) cylindrical element 121 (or its inner and/or outer sides), for example 1.5-8 times, or 2-5 times. For example, a longitudinal extension of the distally tapered portion of connection element 130 may be about 0.5-5 times the longitudinal extension of (generally) cylindrical element 121 (and or its inner and/or outer sides), for example 0.7+3 times, or 0.8-2 times. The same dimensions may apply for the proximally tapered portion of connection element 130.

Although depicted in FIG. 1, dilator shaft 110 does not necessarily include an inner lumen.

FIG. 2 shows a further example for a dilator 200 including a dilator shaft 210, a reinforcing element 220 and a connection element 130.

Dilator shaft 210 may (generally) be similar as described with reference to dilator shaft 110. However, the dilator shaft 210 includes a distal region 211 with a relatively larger diameter and a proximal region 212 with a relatively smaller diameter. In between distal region 211 and proximal region 212 a transition zone 213 (e.g. a shoulder) is provided.

The reinforcing element 220 essentially consists of a (generally) cylindrical element 221 that provides a (generally) cylindrical inner side and a (generally) cylindrical outer side of reinforcing element 220. The reinforcing element 220 includes an opening 226 with an inner diameter essentially identical to an inner diameter of (generally) cylindrical element 221. In other examples, an opening with a smaller diameter may be provided or even no opening may be provided. The reinforcing element 120 may include a (generally) ring-like shape.

An outer diameter of cylindrical element 221 or its (generally) cylindrical face or outer side abuts at an inner diameter of distal region 211. The distal region 211 may be snapped on cylindrical element 221, for example, the distal region 2011 may be shrink fitted, and/or attached by an adhesive. Cylindrical element 221 extends distally from a distal end of dilator shaft 110. For example, the distal extension may have a value in a range of 0.5-5 or 1-3 times an inner radius of cylindrical outer side 221.

The connection element 230 may (generally) include a longitudinal extension and an outer diameter that may be identical as described with reference to connection element 130. The connection element 230 may be injection molded around dilator shaft 110 and reinforcing element 120. Hence, the connection elements inner diameter may follow the outer shape of dilator shaft 110 and reinforcing element 120. Hence, connection element 230 may include a step 231 at transition zone 213. Further, connection element may include a step 232 at the distal end of distal portion 211 of dilator shaft 210. The reinforcing element 220 includes a distal portion 225 at which connection element 230 does not extend.

A longitudinal extension of reinforcing element 210 (or cylindrical element 221 or its cylindrical (inner or outer) face) may include the same values relative to an inner diameter of proximal portion 212 of dilator shaft 210 as outlined with reference to reinforcing element 110 (or cylindrical element 121) and an inner diameter of dilator shaft 110. A longitudinal extension of connection element 230 may include the same values relative to the longitudinal extension of reinforcing element 220 (or cylindrical element 221) as outlined with reference to connection element 130 relative to the longitudinal extension of reinforcing element 120 (or (generally) cylindrical element 121).

A longitudinal extension of distal portion 211 of dilator shaft 210 may include a value of 0.2-1, 0.2-0.9 or 0.3-0.8 of a longitudinal extension of reinforcing element 220 (or cylindrical element 221 (or its inner and/or outer side).

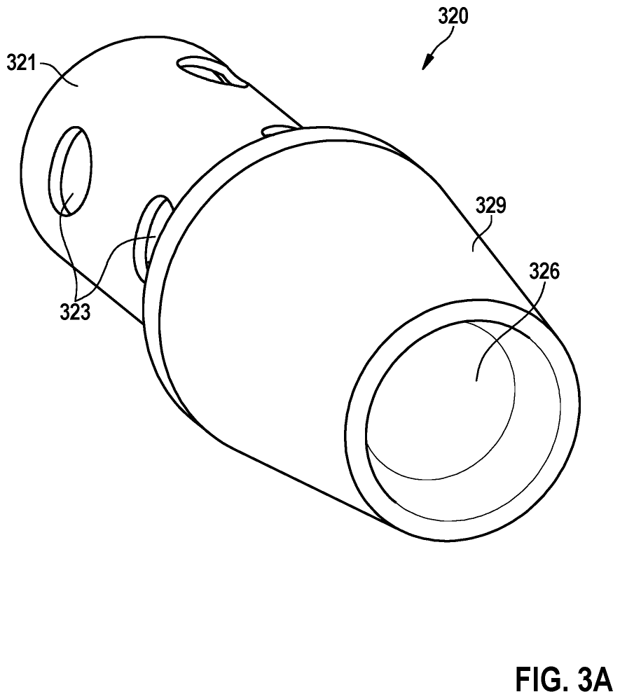

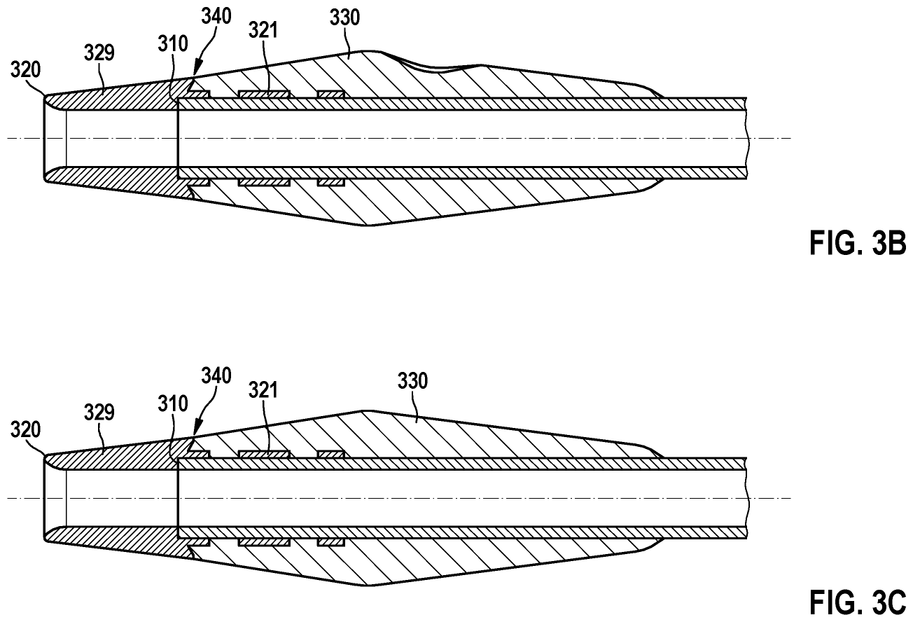

FIG. 3A shows an exemplary reinforcing element 320. The reinforcing element 320 includes a proximal portion 321 and a distal portion 329 being situated distal to the proximal portion 321. The proximal portion 321 has a (generally) cylindrical shape. The (generally) cylindrical proximal portion forms an outer proximal side and an inner proximal side. The proximal portion 321 includes several through holes 323. The distal portion 329 is conical distal portion 329, preferably the distal portion is distally tapered. The distal portion has an outer diameter. The outer diameter of distal portion 329 at its proximal end may be larger than an outer diameter of the (cylindrical) proximal portion 321. The outer diameter of distal portion 329 may taper distally. For example, at a distal end of distal portion 329 its outer diameter may be similar to or slightly larger than an outer diameter of (generally) cylindrical outer side.

The distal portion 329 may include an opening 326. An inner diameter of opening 326 may be identical to an inner diameter of (generally) of the cylindrical proximal portion 321. For example, the reinforcing element 320 may thus include (an inner lumen with) a constant inner diameter. The distal end of distal portion 329 may be rounded (and thus being blunt).

For example, the reinforcing element 320 may be attached to a dilator shaft via a connection element similarly as described with reference to FIGS. 1 and 2. Such a connection element may for example extend into openings 323 and abut at the (generally) proximal portion 321. Such a connection element may for example extend from a proximal direction distally and stop at the proximal end of distal portion 329. The reinforcing element is made of a metal or metal alloy.

FIG. 3B and 3C show a cross-sectional view of the reinforcing element of FIG. 3A reinforcing element being connected to the connection element 330 and the dilator shaft 310. Here it can be seen that the distal portion 329 of the reinforcing element has at its proximal end an undercut 340 which in addition to the through hole provide an anchoring point for the connection element 330.

The longitudinal extension from a distal-most part of the reinforcing element to a proximal-most part of the connection element is within the range of 6.0 to 7.0 mm. The reinforcing element may have a longitudinal extension of 2.5 to 3.5 mm. For example, the distal portion of the reinforcing element may have a length (longitudinal extension) of 1.0 mm to 2.0 mm and an outer diameter of 0.9 to 1.3 mm, and the proximal portion of the reinforcing element may have length (longitudinal extension) of 1.0 mm to 2.0 mm and a smaller outer diameter than the distal portion.

The connection element has at its mid portion an outer diameter of 1.0 mm to 2.0 mm, which is larger than the outer diameter of its distal portion and its proximal portion. The connection element has an inner diameter of 0.4 to 0.6 mm. The dilator shaft has the same inner diameter than the inner diameter of the reinforcing element.

In FIG. 3B an injection point of an injection molding process can be seen for the connection element whereas the connection element 330 in FIG. 3C has no injection point (e.g. because the polymer was connected to the reinforcing element 320 in another way and not by injection molding).

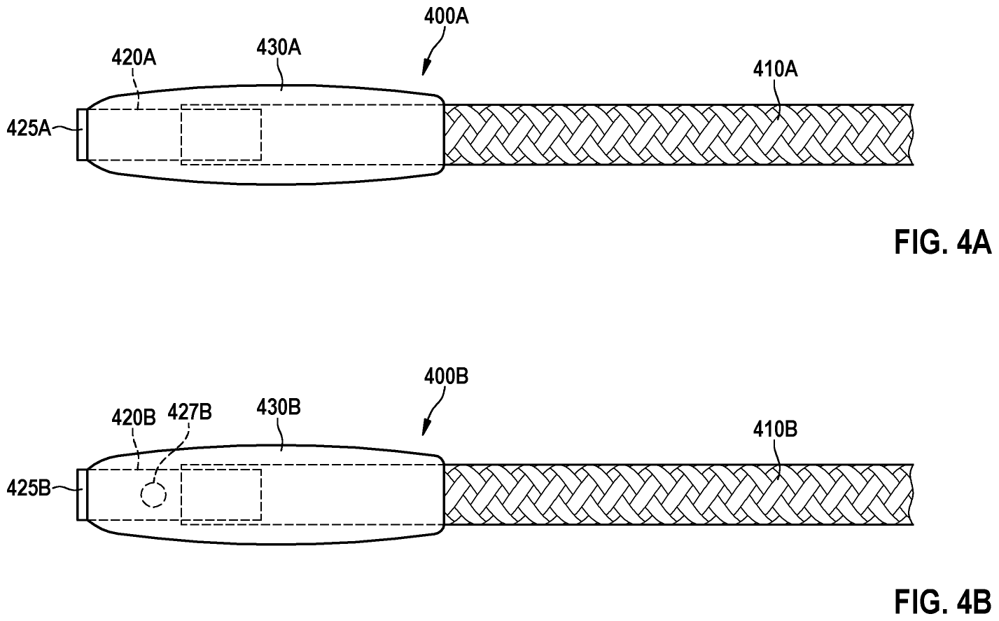

FIGS. 4A and 4B show two examples of dilators 400A and 400B. Dilators 400A and 400B each include a dilator shaft 410A, 410B, respectively, a reinforcing element 420A, 420B, respectively, and a connection element 430A, 430B, respectively. The reinforcing elements 420A and 420B each include a distal portion 425A and 425B, respectively, in which the connection elements 430A and 430B, respectively, do not extend. The dilator shafts 410A and 410B of dilators 400A and 400B may be braided.

The reinforcing element 420B of dilator 400B includes an indentation 427B, into which connection element 430B extends.

The reinforcing element 420A/420B is preferably the reinforcing element shown in FIG. 3 and is made of a metal or metal alloy.

The connection element 430A/430B preferably has an olive shape. The olive shape enables an evenly dispersion of injected contrast agent over the lesion. The connection element is made of a polymer. The mid portion of the olive shape has a smaller diameter than the distal support catheter end.

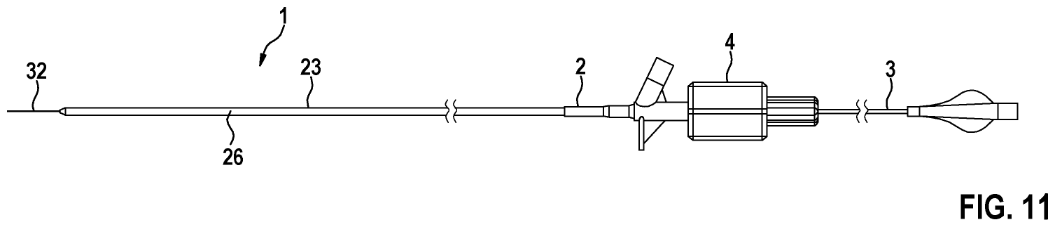

The dilator can be used with/arranged within a (multifunctional) catheter system as describe in FIG. 11. By pulling the dilator back (in proximal direction), due to the olive shape the user has a haptic sensation once the dilator enters the support catheter. This may eliminate a need to perform this pulling back step under X-ray (which means less radiation for the patient).

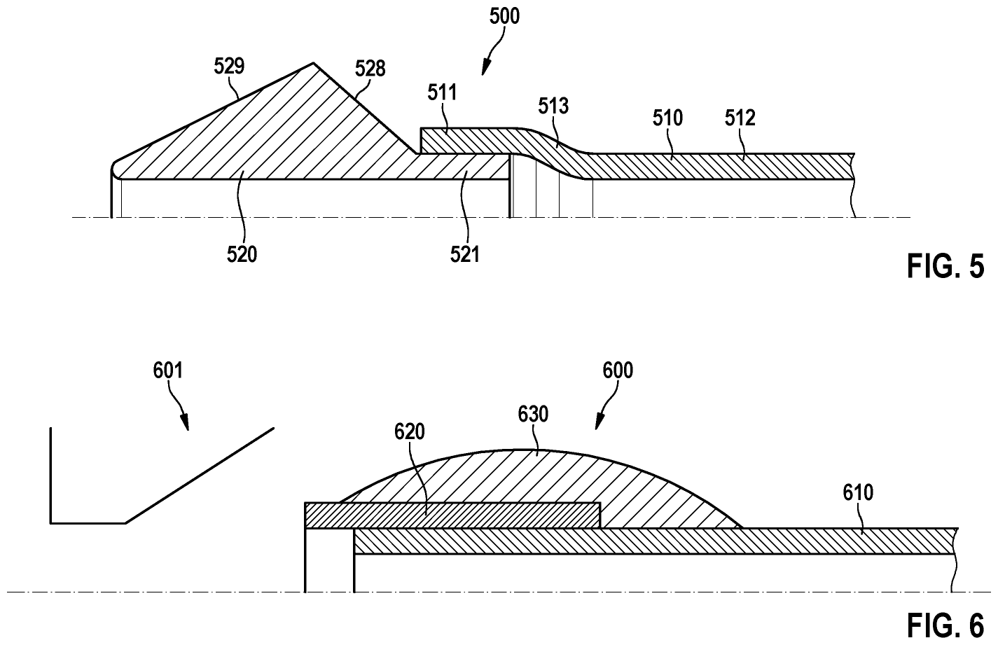

FIG. 5 shows a cross-sectional view of an upper half of a dilator 500 including a dilator shaft 510 and a reinforcing element 520. The reinforcing element 520 may include metal and/or may be manufactured by machining, such as lathing. The reinforcing element 520 may include a shape similar to that outlined with reference to reinforcing element 320 shown in FIG. 3. The reinforcing element 520 may, e.g. include a (generally) cylindrical (outer) face 521, forming a proximal portion of reinforcing element 520. The reinforcing element 520 may further include a distally tapered distal portion 529, and a proximally tapered middle portion 528. By machining, e.g. lathing, reinforcing element 520 may be manufactured in a dimensionally accurate and low cost manner.

A connection element may be absent in dilator 500. Instead, the distal tip of dilator 500 may entirely be formed integrally, e.g. based on a metal material (e.g. by machining such as lathing a metal workpiece). However, distal tip of dilator 500 and/or reinforcing element 520 may in other examples also include other materials, such as ceramics or a polymer.

The dilator shaft 510 may for example include a braided material as described herein.

The dilator shaft 510 may include a proximal portion 512 and a distal portion 511, wherein the proximal portion 512 includes a constant, but smaller diameter, whereas the distal portion 511 includes a constant but larger diameter. In between proximal portion 512 and distal portion 511, a transition zone 513 may be located.

An outer side of reinforcing element 520, more specifically (generally) cylindrical outer face 511, may be attached to an inner side of distal portion 511, for example by clipping on, heat shrinking and/or an adhesive. The (generally) cylindrical outer face 521 may end or abut at transition zone 513.

The reinforcing element 520 may be radiopaque, at least in part. The distal tip of reinforcing element 520 may be rounded to support an atraumatic tip design and, e.g., to prevent damage of a guidewire when inserting the dilator over the guidewire.

FIG. 6 shows the manufacture of a further example dilator 600 in a cross sectional view of an upper half of dilator 600. A s dilator haft 610 is provided that may be adapted as outlined herein, e.g. braided. A radiopaque, e.g. metal, cannula is placed over the dilator shaft 610 such as to form a reinforcing element 620. A connection element 630 is provided as described herein, e.g. by using a polymer covering. The connection element 630 may fix the cannula on the dilator shaft 610. The connection element 630 may for example be provided by injection molding or welding a portion of a tubing.

The geometry of the distal end of dilator 600 may then be formed by a mold 601, e.g. forming a suitable counterpart. Therein, the connection element 630 may help to form rounded edges such as to form an atraumatic tip. By using mold 601, dimensional accuracy, a consistent shape over many samples and low costs may be achieved.

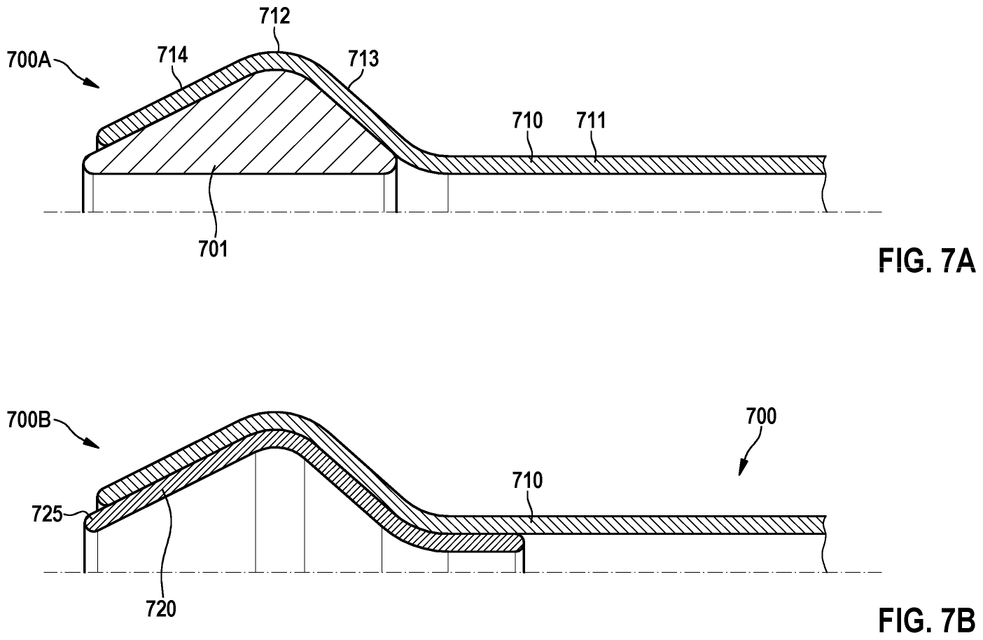

FIG. 7 shows the manufacturing of another example of a dilator 700 in a cross-sectional view. In step 700A, a dilator shaft 710 with a proximal portion 711 and a distal portion 712 is provided. By using a shaping element 701, distal portion 712 is widened, e.g. such as to form a distally tapered portion 714 (arranged distally) and a proximally tapered portion 713 (arranged proximally).

Once the shaping element is removed, in step 700B, reinforcing element 720 is inserted into the dilator shaft 710 such that the reinforcing element 720 includes a distal portion 725. Shaft 710 may not extend over distal portion 725. The reinforcing element 720 may include metal. The reinforcing element 720 may be manufactured by lathing. However, reinforcing element 720 may in other examples also include other materials, such as ceramics or a polymer. A connection between reinforcing element 720 and dilator shaft 710 may be provided by shrink fitting the dilator shaft 710 on reinforcing element 720 and/or by heating the dilator shaft 710, for example.

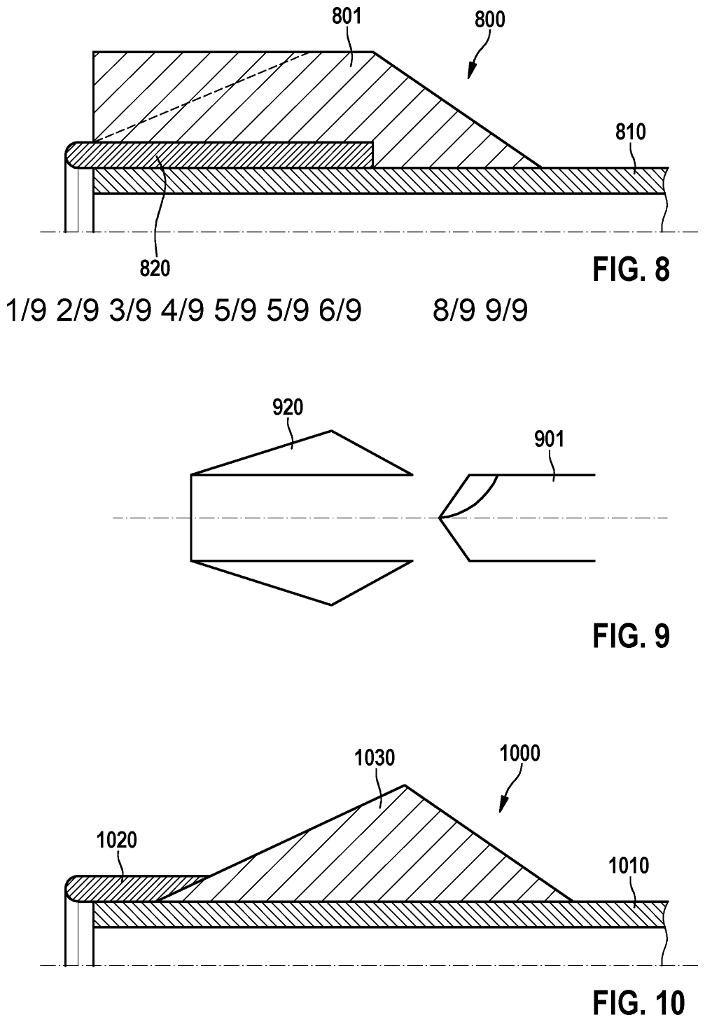

FIG. 8 shows a further aspect of manufacturing a dilator 800 as described herein. FIG. 8 specifically shows a longitudinal cross-section of an upper half of dilator 800. A dilator shaft 810 may be provided. A reinforcing element 820 may be provided at least partly on an outer diameter of the dilator shaft 810. The reinforcing element 820 may be provided by molding or injection molding. The connection element 801 may then be provided, e.g. by molding or injection molding. Similarly as described with reference to FIG. 6, a mold, such as a tip shaping device, may be used to thermally form a desired tip shape.

FIG. 9 shows a further aspect of manufacturing a reinforcing element 920 as described herein. The reinforcing element 920 may include a shape such that it may be used without a connection element on a dilator shaft of a dilator. The reinforcing element may be manufactured by drilling, eroding, laser cutting etc. 901 a cylindrical opening into a bulk part. For example, the bulk part may be manufactured by injection molding, casting, machining, etc. Subsequently, the cylindrical opening may be created. For example, the opening may be in the form of a longitudinal through hole with a uniform inner diameter. However, it is also possible to use varying diameters. E.g. a first inner diameter may be provided by a drilling, eroding, laser cutting, etc. on a distal side, whereas a second inner diameter may be provided similarly on a proximal side. The reinforcing element 920 may be attached to the dilator shaft of a dilator as described herein, e.g. by an adhesive, by pressing, etc.

FIG. 10 shows a further example for manufacturing a dilator 1000 in a cross sectional view of an upper half of dilator 1000. A preformed, e.g. molded, part 1030 may be fitted over the dilator shaft 1010. A reinforcing element 1020, e.g. a (metal) band, which may be a radiopaque marker band, may be swaged at a distal portion of the dilator shaft 110, and overlapping in part with the preformed part 1030. Thus, reinforcing element 1020 may secure the preformed part 1030 on the dilator shaft 1010. The preformed part 1030 may provide the function described herein with reference to connection elements and the preformed part 1030 may in general include the same outer diameter and longitudinal extension. However, part 1030 may not have a connecting function, in this example. Instead, by swaging reinforcing element 1020 into the dilator shaft 1010, also part 1030 may be connected to the dilator shaft 1010.

FIG. 11 shows a catheter system 1, preferably a multifunctional catheter system, including a support catheter or retractable sheath 2, a dilator 3 with a distal dilator end 32 and optionally a (locking) handle 4. The support catheter shaft 23 defines a support catheter lumen 26 capable of receiving the dilator 3. Thus, the dilator 3 is arranged within the support catheter lumen 26 of the support catheter 2. The dilator shaft and/or the support catheter shaft may be reinforced e.g. with a metal braid or a metal braid with interlaced pairs of longitudinally wires. The support catheter may have a straight-edged distal end (which enables a higher pushability and avoids flaring). The support catheter may have at its distal end a radiopaque marker, preferably in form of a metal (alloy) ring or metal (alloy) cap capping the distal support catheter end and having a distal opening and rounded edges.

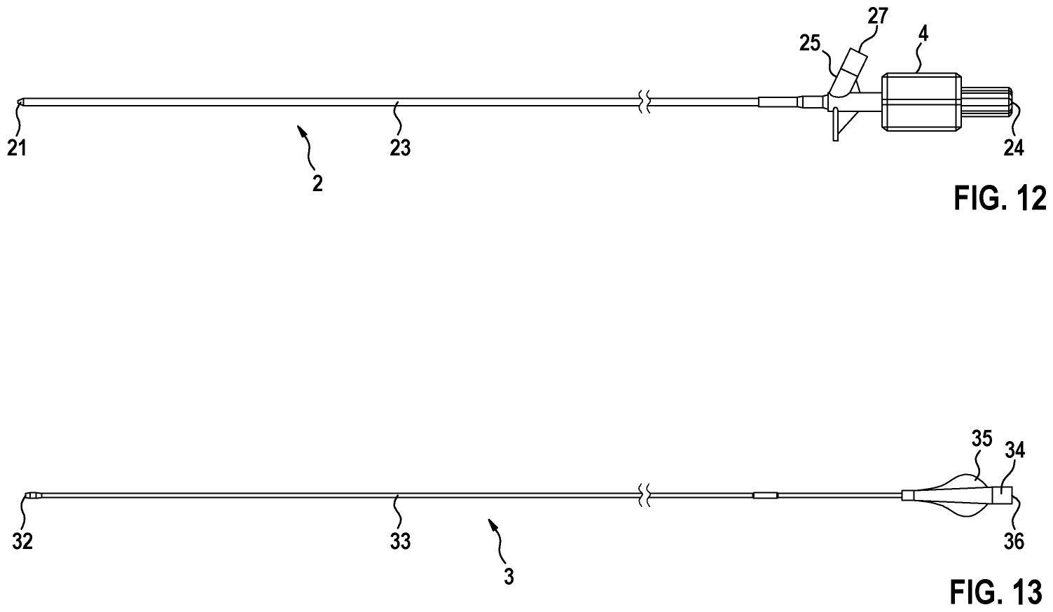

In FIG. 12 a support catheter 2 is shown. The support catheter 2 has a distal support catheter end 21, a proximal support catheter end 24 and a support catheter shaft 23 extending between the support catheter distal end 21 and the support catheter proximal end 24. The proximal support catheter end 24 can includes a locking handle 4 or may be connected to the locking handle 4. The locking handle may have a gripping surface and an ergonomic design. The support catheter may include a manifold which is connected to the locking handle 4. The support catheter 2 may include one or more support catheter ports 27, preferably one or more ports for a fluid medium, e.g. flushing port, inflation and/or deflation port. The distal support catheter end 21 is the end which is inserted into a human or animal body. The proximal outer catheter end 24 is the end, which is operated by an operator, in particular a physician.

FIG. 13 shows a dilator 3 having a distal dilator end 32, a proximal dilator end 34 and a dilator shaft 33 extending between the distal dilator end 32 and the proximal dilator end 34. The proximal dilator end 34 may include a dilator manifold 35. The dilator manifold can have a shape with which the operator, e.g. a physician can easily use it to manipulate the dilator. The dilator may include one or more dilator lumens (not visible). Preferably, the dilator includes a first lumen for receiving a guidewire and a second lumen for receiving a fluid medium, e.g. contrast agent. However, the dilator may have only one lumen enabling at the same time injection of a guidewire and a contrast agent. The dilator, preferably the dilator manifold 35 can include a dilator port 36 for injecting fluid medium (e.g. a contrast agent) into at least one of the dilator lumens.

The dilator shown in FIGS. 11 to 13 is a dilator as described herein, especially one of the dilators described in FIGS. 1 to 4A/B.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A dilator comprising:
a shaft;
a reinforcing element connected to a distal end of the shaft; and
a connection element at least partially enclosing one or both of the shaft and the reinforcing element
wherein the connection element extends into at least one indentation, recess or through hole of the reinforcing element, wherein the connection element consists of a polymer and wherein the reinforcing element consists of a metal or metal alloy.

2. The dilator according to claim 1, wherein the reinforcing element comprises a proximal portion and a conical distal portion situated distally to the proximal portion.

3. The dilator according to claim 2, wherein the reinforcing element extends at least in part distally from a distal end of the connection element.

4. The dilator according to claim 2, wherein the connection element comprises an olive shape.

5. The dilator according to claim 2, wherein the reinforcing element comprises a lumen.

6. The dilator according to claim 2, wherein the connection element at least in part extends around an outer diameter of the shaft and the proximal portion of the reinforcing element and/or the distal portion of the reinforcing element to form a step.

7. The dilator according to claim 1, wherein the reinforcing element at least in part extends around an outer diameter of the shaft.

8. The dilator according to claim 1, wherein the reinforcing element at least in part extends along an inner diameter of the shaft.

9. The dilator according to claim 1, wherein the shaft comprises a distal region and a proximal region and wherein the distal region has a larger diameter than the proximal region.

10. The dilator according to claim 8, wherein the proximal portion of the reinforcing element ends or abuts at a transition zone between the distal region and the proximal region.

11. The dilator according to claim 1, wherein the reinforcing element comprises a proximal portion and the proximal portion comprises at least one indentation, recess or through hole.

12. The dilator according to claim 1, wherein the reinforcing element comprises a distal opening.

13. The dilator according to claim 1, wherein the reinforcing element comprises a distally tapered distal end.

14. The dilator according to claim 1, wherein the shaft comprises a reinforcing braid.

15. (canceled).

16. A catheter system comprising a dilator according to claim 1 and, a support catheter comprising a support catheter shaft defining a support catheter lumen configured to receive the dilator.

17. The catheter system according to claim 16, wherein the support catheter comprises a metal ring at a distal portion of the support catheter.

18. The catheter system according to claim 16, wherein the support catheter shaft is reinforced with one of a metal braid and metal braid with interlaced pairs of longitudinal wires.

19. (canceled).

20. The dilator according to claim 1, wherein the reinforcing element is connected to the distal end of the shaft via one of a molded connection or glue.

21. The dilator according to claim 1, wherein reinforcing element comprises cylindrical proximal portion and a conical distal portion situated distally to the cylindrical proximal portion.
